(19) United States
(12) Patent Application Publication
(10) Pub. No.: US 2026/0076893 A1
(43) Pub. Date: Mar. 19, 2026

(54) SELF-FOAMING CLEANSING COMPOSITION

(71) Applicant: Conopco, Inc., d/b/a UNILEVER, Hoboken, NJ (US)

(72) Inventors: Katelyn Fitzpatrick BARRON, Trumbull, CT (US); Taylor Victoria BRADFORD, Highland, MD (US); Bivash Ranjan DASGUPTA, Cheshire, CT (US); Gabriela Del Rosario PIRRO, Fairfield, CT (US)

(73) Assignee: Conopco, Inc., d/b/a UNILEVER, Hoboken, NJ (US)

(21) Appl. No.: 19/110,575

(22) PCT Filed: Sep. 11, 2023
(86) PCT No.: PCT/EP2023/074860

§ 371 Date: Mar. 11, 2025

Related U.S. Application Data
(60) Provisional application No. 63/407,388, filed on Sep. 16, 2022.

(30) Foreign Application Priority Data
Nov. 4, 2022 (EP) 22205506.3

(51) Int. Cl. A61K 8/92; A61K 8/34; A61K 8/36; A61K 8/365; A61K 8/44; A61K 8/60; A61K 8/67; A61Q 19/00; A61Q 19/10

(52) U.S. Cl. CPC A61K 8/922; A61K 8/345; A61K 8/361; A61K 8/365; A61K 8/442; A61K 8/60; A61K 8/678; A61Q 19/007; A61Q 19/10; A61K 2800/30; A61K 2800/51; A61K 2800/596

(57) ABSTRACT

A self-foaming cleansing composition comprises 2.5 to 15% by weight of a surfactant based on the total weight of the self-foaming cleansing composition, wherein the surfactant comprises an anionic surfactant and a zwitterionic surfactant; a humectant; and 0.01 to 1.0% by weight of an emollient based on the total weight of the self-foaming cleansing composition.

### FIELD OF THE INVENTION

Disclosed herein are self-foaming cleansing compositions. The self-foaming cleansing compositions include a surfactant, a humectant, an emollient, and a chelator.

### BACKGROUND OF THE INVENTION

Personal cleansing compositions are popular for their ability to remove dirt, oil and debris from the skin or hair of the consumer. Typical surfactant-based cleansers are formulated to provide a specific cleansing power, feel, mildness and lather volume. Each criterion is significant in the desirability of the cleansing product. For example, lather volume is significant as one factor in determining the efficacy and reach of the product. Moreover, personal cleansers should gently cleanse the skin or hair of the user without causing undesirable irritation or leaving the skin dry. Other ingredients may be added to the surfactant-based composition to aid in the cleansing process.

Liquid cleansing compositions often contain benefit agents also known as actives. Such benefit agents or actives are often included in liquid cleansing compositions with an intention to deliver a benefit. For example, a moisturizing agent can be included in cleansing compositions such as a handwash composition or a bodywash composition, to deliver a moisturizing effect.

However, it can be difficult to achieve the intended benefit from the active or benefit agent in a cleansing composition for several reasons. One reason being that deposition of an active on a surfactant to be cleansed or washed is difficult as the cleansing action generally includes a step of removing the composition (e.g., washing it off) from the surface, typically within a few seconds to a few minutes after application. Therefore, depositing an active on to a surface in a limited duration can be very challenging.

There is always a need to provide skin cleansing formulations having desirable cleansing efficacy without being harsh, without causing problems to the skin, and having the ability to deliver benefit agents such as moisturizers, antibacterial actives, etc.

Environmentally friendly cleansing compositions are continually desired. Accordingly, it can furthermore be desirable to provide a cleansing composition that is biodegradable and one that is vegan or devoid of animal products or by-products.

International Patent Publication No. WO 03/074020 A1 discloses an ordered liquid crystalline phase cleaning composition that contains particles with a structure comprising a benefit agent and a gelation agent and provides high foaming within a preferred embodiment level of free emollient equal to or in excess of the level of surfactant. A method of depositing a benefit agent to the skin or hair with the ordered liquid crystalline phase cleansing composition is also disclosed.

U.S. Pat. No. 9,724,283 B2 discloses an aqueous hair cleansing composition comprising (a) at least alkyl poly glucoside; (b) at least one betaine compound; and (c) at least one acyl amino acid, wherein the composition is sulfate free.

### SUMMARY OF THE INVENTION

Disclosed in various aspects are self-foaming cleansing compositions. The self-foaming cleansing compositions

A self-foaming cleansing composition comprises 2.5 to 15% by weight of a surfactant based on the total weight of the self-foaming cleansing composition, wherein the surfactant comprises an anionic surfactant and a zwitterionic surfactant; a humectant; and 0.01 to 1.0% by weight of an emollient based on the total weight of the self-foaming cleansing composition.

These and other features and characteristics are more particularly described below.

### DETAILED DESCRIPTION OF THE INVENTION

Disclosed herein is a self-foaming cleansing composition. The self-foaming cleansing composition comprises a surfactant comprising an anionic surfactant and a zwitterionic surfactant, a humectant, and an emollient. It was unexpectedly found that this self-foaming cleansing composition is able to deposit the emollient on a user's skin thereby giving the user's skin a moisturizing, hydrating effect even though the cleansing composition is rinsed from the user's skin after a short amount of time (e.g., 10 seconds to 5 minutes). The self-foaming cleansing compositions also provide anti-inflammatory properties to the user's skin. Additionally, the self-foaming cleansing composition can be made sulfate free. Sulfate free as referred to herein means that the composition is free from sulfate based surfactants, for example, sodium laureth sulfate (SLS) or sodium pareth sulfate (SPES). Sulfate free compositions are desirable as being more environmentally friendly, mild, and have a desirable sensorial experience. The self-foaming cleansing composition can also be made with biodegradable ingredients such that the entire composition is biodegradable. The self-foaming compositions can be vegan such that the compositions are completely devoid of any animal product or any animal by-products.

The self-foaming cleansing compositions disclosed herein is completely biodegradable. In embodiments, the self-foaming cleansing compositions contain moringa seed oil and/or tocopherol (Vitamin E). It was unexpectedly found that the self-foaming cleansing compositions containing moringa seed oil in the amounts disclosed herein were active on the user's skin after use. In living skin assays, it was found that the use of moringa seed oil increased the gene expression in proteins responsible for natural moisturization, skin hydration, inflammation, and skin barrier lipids as compared to a composition not containing moringa seed oil. Accordingly, in a further aspect, the invention relates to the use of moringa seed oil to enhance expression of genes selected from the group consisting of CASP14, ASP3, IL1-α, HAS3, CERS, ELOVL2 and combinations thereof. Preferably to the use of moringa oil to enhance expression of the genes selected from the group consisting of CASP14, ASP3, IL1-α, and combinations thereof. Preferably, such upregulation is manifested by application to the skin via a cleansing composition of the invention. The invention further relates to the use of moringa seed oil to increase self-moisturisation of the skin. Self-moisturisation means without the presence of externally applied moisturiser e.g. to close-off the surface, like oils or without the presence of e.g. humectants, but preferably via upregulation of genes, such as those involved in skin moisturisation, such as for example CASP14. Preferably, such upregulation is manifested by application to the skin via a cleansing composition of the invention. The invention further relates to the use of moringa seed oil to increase self-hydration of the skin. Self-hydration means without the presence of externally applied moisturiser, e.g. to close-off the surface, like oils or without the presence of e.g. humectants, but preferably via upregulation of genes, such as those involved in skin hydration, such as for example ASP3, HAS3, or of genes related to barrier lipids such as CERS or ELOV2. Preferably, such upregulation is manifested by application to the skin via a cleansing composition of the invention. The invention further relates to the use of moringa seed oil to auto-decrease inflammation of the skin. Auto-decrease of inflammation means without the presence of externally applied anti-inflammatory agents, but via upregulation of genes, such as those involved in anti-inflammation, such as for example IL1-α. Preferably, such upregulation is manifested by application to the skin via a cleansing composition of the invention. In this respect, the invention can relate to moringa seed oil for use in decreasing inflammation of the skin.

Accordingly, the invention further relates to a cleansing composition comprising moringa seed oil. It can be preferred, that such a cleansing composition further comprises tocopherol. In a preferred embodiment, such a cleansing composition is the self-foaming cleansing composition of the present invention.

Replacement of non-biodegradable ingredients (e.g., tetrasodium EDTA) with biodegradable ingredients (e.g., sodium gluconate) can ensure the self-foaming cleansing composition is biodegradable.

To add antioxidation properties to the self-foaming cleansing composition, tocopherol (Vitamin E) can be added. Antioxidation can be measured using a DPPH scavenging method where DPPH (1,1-Diphenyl-2-picrylhydrazyl) is a stable free radical. On accepting hydrogen from a corresponding donor, its solutions lose a characteristic deep purple color and antiradical activity of tested formulations can be expressed as a percentage decrease of DPPH concentration. It was unexpectedly found that when the self-foaming cleansing compositions were tested at varying ethanol dilutions 0.1% (0.1% formula, 99.9% ethanol) to 50% (50% formula, 50% ethanol), there was a dose-dependent response to antioxidant activity. When dosed with tocopherol (Vitamin E) at 0.025% by weight and 0.05% by weight, the self-foaming cleansing compositions were found to carry statistically significant free radical reduction at ethanol dilutions down to 10%. Accordingly, in a further aspect, the invention relates to the use of tocopherol in a composition according to the invention to provide anti-oxidant activity to the cleansing composition, preferably dose-dependent anti-oxidant activity. The invention relates further to the use of tocopherol to reduce free radicals in a cleansing composition according to the invention, preferably dose-dependent anti-oxidant activity. In a further embodiment, it may be preferred that the cleansing composition of the invention comprises tocopherol and moringa seed oil.

The self-foaming cleansing composition can include the surfactant in an amount of 2.5 to 15% by weight based on the total weight of the self-foaming cleansing composition, for example, 2.5 to 10% by weight, based on the total weight of the self-foaming cleansing composition, for example, 3.0 to 8% by weight, based on the total weight of the self-foaming cleansing composition, for example, 3.5 to 7.5% by weight, based on the total weight of the self-foaming cleansing composition, including any and all ranges and values subsumed therein. The surfactant can comprise an anionic surfactant, an amphoteric surfactant, a zwitterionic surfactant, a nonionic surfactant, a cationic surfactant, or a combination thereof. In an embodiment, the surfactant can comprise an anionic surfactant and a zwitterionic surfactant. In an embodiment, the surfactant is an anionic surfactant and a zwitterionic surfactant.

The surfactant present in the self-foaming cleansing composition can include an anionic surfactant and an amphoteric surfactant present in a ratio of 0.5:2, for example, 0.75 to 1.5, for example, 0.8 to 1, for example, 0.85 to 0.95. In an embodiment, the ratio of anionic surfactant to amphoteric surfactant is 0.5:2, preferably 0.75 to 1.5, more preferably 0.8 to 1, even more preferably 0.85 to 0.95.

The anionic surfactant can include aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic surfactant may also be alkyl sulfosuccinates (including mono- and dialkyl, e.g., C₆-C₂₂ sulfosuccinates); alkyl and acyl taurates (often methyl taurates), alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:

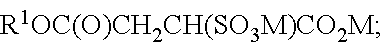

and amide-MEA sulfosuccinates of the formula:

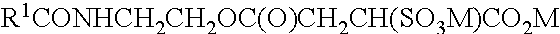

wherein R¹ ranges from C₈-C₂₂ alkyl.

Sarcosinates are generally indicated by the formula:

R²CON(CH₃)CH₂CO₂M, wherein R² ranges from C₈-C₂₀ alkyl.

Taurates are generally identified by formula:

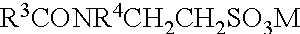

wherein R³ is a C₈-C₂₀ alkyl, R⁴ is a C₁-C₄ alkyl.

M is a solubilizing cation as previously described.

The self-foaming cleansing composition disclosed herein may contain C₈-C₁₈ acyl isethionates.

These esters are prepared by a reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

The acyl isethionate may be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference. This compound has the general formula:

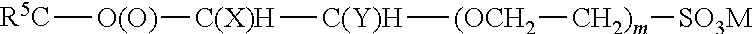

wherein R⁵ is an alkyl group having 8 to 18 carbons, m is an integer from 1 to 4, X and Y are each independently hydrogen or an alkyl group having 1 to 4 carbons and M is a solubilizing cation as previously described.

In an aspect of the self-foaming cleansing composition, the anionic surfactant used is sodium lauroyl glycinate, sodium cocoyl glycinate, sodium lauroyl glutamate, sodium cocoyl glutamate, sodium lauroyl isethionate, sodium cocoyl isethionate, alpha olefin sulfonate (AOS), or a combination thereof. Such anionic surfactants are commercially available from suppliers like Galaxy Surfactants, Clariant, Sino Lion, Stepan Company, and Innospec. In an embodiment, the self-foaming cleansing composition is essentially free of sulfate. Substantially free or essentially free as used herein refers to an amount of less than or equal to 1% by weight, for example, less than or equal to 0.5% by weight, for example, less than or equal to 0.25% by weight, for example, less than or equal to 0.1% by weight, for example, less than or equal to 0.01% by weight, for example, 0% by weight.

Optionally, amphoteric surfactants can be included in the cleansing compositions disclosed herein. Amphoteric surfactants (which depending on pH can be zwitterionic) include sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of amphoteric surfactants include sodium lauroamphoacetate, sodium cocoamphoacetate, or a combination thereof.

As to the zwitterionic surfactants employed in the present cleansing composition, such surfactants include at least one acid group. Such an acid group may be a carboxylic or a sulphonic acid group. They often include quaternary nitrogen, and therefore, can be quaternary amino acids. They should generally include an alkyl or alkenyl group of 7 to 18 carbon atoms and generally comply with an overall structural formula:

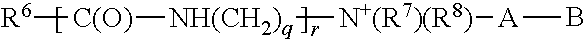

where R⁶ is alkyl or alkenyl of 7 to 18 carbon atoms; R⁷ and R⁸ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

Desirable zwitterionic surfactants for use in the cleansing composition disclosed herein and within the above general formula include simple betaines of formula:

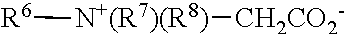

and amido betaines of formula:

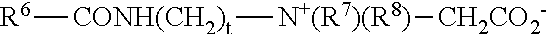

where t is 2 or 3.

In both formulae R⁶, R⁷ and R⁸ are as defined previously. R⁶ may, in particular, be a mixture of C₁₂ and C₁₄ alkyl groups derived from coconut oil so that at least half, preferably at least three quarters of the groups R⁶ have 10 to 14 carbon atoms. R⁷ and R⁸ are preferably methyl.

A further possibility is that the zwitterionic surfactant is a sulphobetaine of formula:

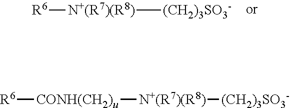

where u is 2 or 3, or variants of these in which —(CH₂)₃SO₃⁻ is replaced by —CH₂C(OH)(H)CH₂SO₃⁻.

In these formulae, R⁶, R⁷ and R⁸ are as previously defined.

Illustrative examples of the zwitterionic surfactants desirable for use include betaines such as lauryl betaine, betaine citrate, cocodimethyl carboxymethyl betaine, cocoamidopropyl betaine, coco alkyldimethyl betaine, and laurylamidopropyl betaine. An additional zwitterionic surfactant suitable for use includes cocoamidopropyl sultaine, for example, cocamidopropyl hydroxysultaine. Preferred zwitterionic surfactants include lauryl betaine, betaine citrate, sodium hydroxymethylglycinate, (carboxymethyl) dimethyl-3-[(1-oxododecyl) amino]propylammonium hydroxide, coco alkyldimethyl betaine, (carboxymethyl) dimethyloleylammonium hydroxide, cocoamidopropyl betaine, (carboxymethyl) dimethyloleylammonium hydroxide, cocoamidopropyl betaine, (carboxylatomethyl) dimethyl(octadecyl)ammonium, cocamidopropyl hydroxysultaine, or a combination thereof. Most preferred zwitterionic surfactants include cocodimethyl carboxymethyl betaine, cocamidopropyl betaine, laurylamidopropyl betaine, cocamidopropyl hydroxysultaine, lauryl hydroxysultaine, cocamide monoethanolamide, or a combination thereof. Such surfactants are made commercially available from suppliers like Stepan Company, Solvay, Evonik and the like and it is within the scope of the cleansing compositions disclosed herein to employ mixtures of the aforementioned surfactants.

Nonionic surfactants may optionally be used in the cleansing composition. When used, nonionic surfactants are typically used at levels as low as 0.5, 1, 1.5 or 2% by weight and at levels as high as 6, 8, 10 or 12% by weight, including any and all ranges and values subsumed therein. The nonionic surfactants which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₃) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

In an aspect, nonionic surfactants can include fatty acid/alcohol ethoxylates having the following structures a) HOCH₂(CH₂)ₛ(CH₂CH₂O)_{c} H or b) HOOC(CH₂)ᵥ(CH₂CH₂O)_{d} H; where s and v are each independently an integer up to 18; and c and d are each independently an integer from 1 or greater. In an aspect, s and v can be each independently 6 to 18; and c and d can be each independently 1 to 30. Other options for nonionic surfactants include those having the formula HOOC(CH₂)ᵢ—CH═CH—(CH₂)ₖ(CH₂CH₂O)_{z} H, where i, k are each independently 5 to 15; and z is 5 to 50. In another aspect, i and k are each independently 6 to 12; and z is 15 to 35.

The nonionic surfactant may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Illustrative examples of nonionic surfactants that can optionally be used in the self-foaming cleansing compositions disclosed herein include, but are not limited to, polyglycoside, cetyl alcohol, decyl glucoside, lauryl glucoside, octaethylene glycol monododecyl ether, n-octyl beta-d-thioglucopyranoside, octyl glucoside, oleyl alcohol, polysorbate, sorbitan, stearyl alcohol, or a combination thereof.

In an aspect, cationic surfactants may optionally be used in the self-foaming cleansing composition of the present application.

One class of cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants for use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and stearyl amidopropyl dimethylamine lactate.

Still other useful cationic surfactants include quaternized hydrolysates of silk, wheat, and keratin proteins, and it is within the scope of the cleansing composition to use mixtures of the aforementioned cationic surfactants.

If used, cationic surfactants will make up no more than 1.0% by weight of the cleansing composition. When present, cationic surfactants typically make up from 0.01 to 0.7%, and more typically, from 0.1 to 0.5% by weight of the cleansing composition, including any and all ranges and values subsumed therein.

The self-foaming cleansing composition further comprises a humectant. The humectant can be present in an amount of 0.01 to 85% by weight, based on the total weight of the self-foaming cleansing composition, for example, 5.0 to 75% by weight, based on the total weight of the self-foaming cleansing composition, for example, 7.5 to 50% by weight, based on the total weight of the self-foaming cleansing composition, for example, 10% to 25% by weight, based on the total weight of the self-foaming cleansing composition, including any and all ranges and values subsumed therein.

The humectant can be employed to assist in moisturization effects of the self-foaming cleansing composition. Humectants are generally known as moisturizers that attract water from the air or deeper in the skin. Stated another way, humectants draw water into the skin, hair, or nails. The humectants can generally be polyhydric alcohol type materials. Typical polyhydric alcohols include glycerol (i.e., glycerine or glycerin), propylene glycol, dipropylene glycol, polypropylene glycol (e.g., PPG-9), polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol, or a combination thereof. Most preferred is glycerin, propylene glycol, dipropylene glycol, or a combination thereof. In an embodiment, the humectant can be propylene glycol, butylene glycol, dipropylene glycol, glycerin, triethylene glycol, erythritol, capryl glycol, hyaluronic acid, polypropylene glycol-7 propyl heptyl ether, or a combination thereof. In an embodiment, the humectant is glycerine.

The self-foaming cleansing composition can further comprise a chelator. Chelation is a type of bonding of ions and molecules to metal ions. The chelator assists in preventing fragrance and color degradation as well as boosting preservative activity.

The self-foaming cleansing composition can also comprise a chelator in an amount of 0.01 to 1.0% by weight, based on the total weight of the self-foaming cleansing composition, for example, 0.05 to 0.75% by weight, based on the total weight of the self-foaming cleansing composition, for example, 0.075 to 0.5% by weight, based on the total weight of the self-foaming cleansing composition, for example, 0.09 to 0.1% by weight, based on the total weight of the self-foaming cleansing composition, including any and all ranges and values subsumed therein.

In an embodiment, the chelator comprises ethylene diaminetetraacetic acid (EDTA), diethylenetriaminepentaacetic acid (DTPA), pentasodium diethylenetriaminepentaacetate, trisodium N-(hydroxyethyl)-ethylenediaminetracetate, an acid form of ethylene diaminetetraacetic acid (EDTA), phytic acid, or a combination thereof. In an embodiment, the chelator is ethylene diaminetetraacetic acid (EDTA), diethylenetriaminepentaacetic acid (DTPA), pentasodium diethylenetriaminepentaacetate, trisodium N-(hydroxyethyl)-ethylenediaminetracetate, an acid form of ethylene diaminetetraacetic acid (EDTA), phytic acid, or a combination thereof.

In an embodiment, the chelator comprises sodium gluconate, nitrilotriacetic acid (NTA), ethylene diamine disuccinic acid (EDDS), iminodisuccinic acid (IDS), salts of methylglycinediacetic acid, methylglycinediacetic acid (MGDA), L-glutamic acid N,N-diacetic acid (GLDA), ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), sodium citrate, or a combination thereof. In an embodiment, when the chelator comprises these chelators, the self-foaming cleansing composition is biodegradable. Accordingly, the composition comprises preferably less than 0.5 wt. %, preferably less than 0.01 wt. % EDTA, and is most preferably free from EDTA.

The composition can also be biodegradable without the use of any chelators. If chelators are to be used and a biodegradable composition is desired, the aforementioned chelators can be utilized.

When using the chelators that are biodegradable, it can be important to ensure the composition is mixed adequately so that the contents are homogenous because these chelators are generally solids at room temperature as compared to the non-biodegradable chelators, which are generally liquids at room temperature. Room temperature as used herein generally refers to a temperature of about 20° C.

The self-foaming cleansing composition can further comprise a nourishing agent. The nourishing agent assists replenishing skin essential nutrients to help skin retain its natural moisture after use of the self-foaming cleansing composition.

The self-foaming composition can further comprise a nourishing agent. The nourishing agent can be present in an amount of 0.01 to 1.0% by weight, based on the total weight of the self-foaming cleansing composition, for example, 0.05 to 0.75% by weight, based on the total weight of the self-foaming cleansing composition, for example, 0.1 to 0.5% by weight, based on the total weight of the self-foaming cleansing composition, for example, 0.2 to 0.3% by weight, based on the total weight of the self-foaming cleansing composition, including any and all ranges and values subsumed therein.

The nourishing agent can comprise a number of materials, including, but not limited to, octadecanoic acid, stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate, or a combination thereof.

Preferably, the nourishing agent comprises C12-C22 fatty acids, preferably, the nourishing agent comprises 10-hydroxystearic acid, 12-hydroxystearic acid, petroselinic acid, conjugated linoleic acid, lauric acid, myristic acid, palmitic acid, stearic acid, behenic acid, or a combination thereof. More preferably, the nourishing agent is 10-hydroxystearic acid, 12-hydroxystearic acid, petroselinic acid, conjugated linoleic acid, lauric acid, myristic acid, palmitic acid, stearic acid, behenic acid, or a combination thereof. Most preferably, the nourishing agent is 12-hydroxystearic acid.

The self-foaming cleansing composition can further comprise an emulsifier. Emulsifiers having an HLB of greater than 8 can be used. Illustrative examples include sorbitan esters such as TWEEN™ 40, TWEEN™ 60, TWEEN™ 80, TWEEN™ 20 (polysorbate 20), or a combination thereof. Typically, emulsifiers for water continuous systems make up from 0.3 to 2.5% by weight of total weight of the self-foaming cleansing composition, including any and all ranges and values subsumed therein.

The self-foaming cleansing composition can further comprise a pH adjusting agent. The pH of the concentrated conditioning composition can be 2 to 9, preferably, 4 to 9, and most preferably, 5 to 6, including any and all ranges and values subsumed therein. Adjusters to modify/buffer the pH can be used. Such pH adjusters include triethylamine, NaOH, KOH, H₂SO₄, HCl, C₆H₈O₇ (i.e., citric acid), C₃H₆O₃ (i.e., lactic acid), or a combination thereof. The pH adjusters are added at amounts to yield the desired final pH. The pH adjusters can include various alpha-hydroxy acids or a combination thereof. For example, the pH adjusters can comprise the alpha-hydroxy acids of citric acid, glycolic acid, lactic acid, malic acid, tartaric acid, or a combination thereof. The pH values can be assessed with commercial instrumentation such as a pH meter made commercially available from Thermo Scientific®. The pH adjusters can be present in an amount of 0.1 to 1.0% by weight of the total weight of the self-foaming cleansing composition, for example, 0.25 to 0.75% by weight, for example, 0.5 to 0.6% by weight of the total weight of the concentrated conditioning composition, including any and all ranges and values subsumed therein.

Preservatives can desirably be incorporated into the self-foaming cleansing composition to protect against the growth of potentially harmful microorganisms. Preservatives are antimicrobial ingredients added to maintain the microbiological safety of products. They act to inhibit the growth of microbes and so reduce the level of microbial contamination. As personal wash formulations such as those disclosed herein contain biodegradable ingredients, they can become unpleasant and unsafe if microbial breakdowns is not controlled. Microbial growth is water dependent, so preservatives must partition to some extent into the aqueous phase of a formulation. Commonly used preservatives can be categorized into the following five classes:
1) Parabens such as Methyl-, Propyl-, and Butylparaben and Germaben II are derived from para-hydroxy benzoic acid. These materials are economical and effective against fungals and some Gram negative bacteria but need a second ingredient to control Gram positives. They also tend to partition more towards the oil phase in emulsion-containing formulations. They are widely employed at levels of 0.01-0.3% by weight and are generally considered safe—though there have been concerns over possible estrogenic activity and links to cancer.
2) Formaldehyde Releasers such as Germall Plus, DMDM Hydantoin, and Imadozolidinyl or Diazolidinyl Urea. This class of materials is effective against bacteria but offers only weak antifungal activity. They are used at levels of 0.1-0.5% by weight in the pH range 3-8. The low levels of free formaldehyde released ensure microbial inhibition, but cause concerns as potential carcinogens.
3) Isothiazolinones such as methylcholoroisothiazolinone (MCI), methylisothiazolinone (MI), and Kathon. Isothiazolinones offer broad spectrum effectiveness over a broad pH range, but they may cause skin irritation for some consumers. This class of materials is employed at low levels, on the order of 10's of ppms.
4) Phenoxyethanol, marketed as Optiphen or Optiphen Plus and Neolone PH 100. Phenoxyethanol is often considered as a milder alternative to parabens or formaldehyde-donors but has a narrow spectrum of applicability to Gram negative bacteria. It is generally combined with caprylyl glycol, sorbic acid/potassium sorbate, or EDTA to create broad spectrum efficacy. It is applicable over a wide range of pH, with a typical usage level of 1% or less. However, there are some concerns over possible carcinogenic activity.
5) Organic Acids such as Benzoic Acid/Sodium Benzoate, Sorbic Acid/Potassium Sorbate, Salicylic Acid/Sodium Salicylate, and Levulinic or Anisic Acids. The use of these acids is confined to aqueous applications in the pH range 2-6. They typically are used at higher levels than some of the above alternatives and have somewhat weaker efficiency against bacteria (which can be augmented by combination with diazolidinyl urea), though they are very good against fungi. This class of preservatives are generally considered as natural.

Preservatives for use in the cleansing compositions disclosed herein preferably include organic-acid based preservatives, preferably sodium benzoate, caprylyl glycol, or a combination thereof. Traditional preservatives for use include hydantoin derivatives and propionate salts.

Other preservatives for use are iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin and benzyl alcohol and mixtures thereof. Other preservatives include sodium benzoate, sodium dehydroacetate, chlorophenesin, decylene glycol, methylchloroisothiazolinone, methylisothiazolinone, or a combination thereof. The preservatives should be selected having regard for the use of the composition and possible incompatibilities between the preservatives and other ingredients in the cleansing composition. Also preferred is a preservative system with hydroxyacetophenone alone or in a mixture with other preservatives. Particularly preferred is sodium benzoate.

The self-foaming cleansing compositions disclosed herein typically contain water in an amount of 20 to 90% by weight, more particularly 50 to 85% by weight, based on the total weight of the self-foaming cleansing composition, including any and all ranges and values subsumed therein. Such water contents are representative of a relatively broad range of compositions, including both concentrated and non-concentrates products, with formulations having water contents of 20 to less than 50% by weight of water being typical of concentrated products.

In use, the self-foaming cleansing compositions are commonly diluted with water. The extent of dilution depends on the particular product form. Less commonly, but also contemplated for use herein are formulations that are foamed without dilution, typically through the use of pump dispensers in which product is passed through a screen in the pump.

The cleansing composition can additionally include various additives including, but not limited to, colorants, anti-dandruff agents, skin feel agents, hair dyes, styling polymer, silicon oil, cationic polymers, or a combination thereof. Each of these substances be present in an amount of 0.03 to 5%, preferably between 0.1 and 3% by weight of the total weight of the self-foaming cleansing composition, including any and all ranges and values subsumed therein. For example, when present, colorants can be present in an amount of 5 parts per million (ppm) to 15 ppm, for example, about 15 ppm.

Additional optional ingredients which may be present in the self-foaming cleansing compositions are, for example: fragrances; coloring agents; opacifiers and pearlizers such as zinc stearate, magnesium stearate, TiO₂, ethylene glycol monostearate (EGMS), ethylene glycol distearate (EGDS) or Lytron 621 (Styrene/Acrylate copolymer) and the like; antioxidants, for example, butylated hydroxytoluene (BHT) and the like; stabilizers; suds boosters, such as for example, coconut acyl mono- or diethanol amides; ionizing salts, such as, for example, sodium chloride and sodium sulfate, and other ingredients such as are conventionally used in liquid soap formulations. The total amount of such additional optional ingredients is typically 0 to 10% by weight, more particularly from 0.1 to 5% by weight, based on the total weight of the self-foaming cleansing composition.

The compositions typically include one or more skin benefit agents. The term “skin benefit agent” is defined as a substance which softens or improves the elasticity, appearance, and youthfulness of the skin (stratum corneum) by either increasing its water content, adding, or replacing lipids and other skin nutrients, or both, and keeps it soft by retarding the decrease of its water content. Included among the skin benefit agents are emollients, including, for example, hydrophobic emollients, hydrophilic emollients, or blends thereof.

The self-foaming cleansing composition further comprises an emollient. The emollient can be present in an amount of 0.01 to 1.0% by weight, based on the total weight of the self-foaming cleansing composition, for example, 0.05 to 0.75% by weight, based on the total weight of the self-foaming cleansing composition, for example, 0.1 to 0.5% by weight, based on the total weight of the self-foaming cleansing composition, for example, 0.2 to 0.4% by weight, based on the total weight of the self-foaming cleansing composition, for example, 0.2 to 0.3% including any and all ranges and values subsumed therein.

In an embodiment, the emollient comprises an oil, a butter, a wax, or a combination thereof. The oil can be Baobab seed oil, Argan kernel oil, behenyl neopentanoate, Borage seed oil, Rapeseed seed oil, Tamanu seed oil, False Flax seed oil, Camellia seed oil, caprylic/capric triglyceride, Safflower seed oil, coco-caprylate/caprate, Coconut oil, Hazel seed oil, Crambe seed oil, Cotton seed oil, Sunflower seed oil, Sea Buckthorn oil, isopropyl myristate, isononyl isonanonoate, isopropyl palmitate, jojoba esters, lanolin oil and lanolin derivatives, Limnanthes Alba (Meadowfoam) seed oil, Linseed seed oil, Macadamia seed oil, Moringa seed oil, Evening Primrose oil, Olive fruit oil, Babassu seed oil, Rice germ oil, Avocado oil, Sacha inchi seed oil, Sweet Almond oil, Castor seed oil, Rosehip seed oil, Chia seed oil, Sage oil, Mongongo kernel oil, Marula seed oil, Sesame seed oil, Sal seed oil, silicone, Jojoba seed oil, squalane, Thyme oil, algae oil, Wheat germ oil, Grapeseed oil, Corn oil, or a combination thereof. In an embodiment, the cleansing composition comprises Moringa seed oil. This may preferably be present in an amount of from 0.01 to 1.0% by weight, based on the total weight of the self-foaming cleansing composition, for example, more preferably, from 0.05 to 0.75% by weight, based on the total weight of the self-foaming cleansing composition, even more preferably from 0.1 to 0.5% by weight, based on the total weight of the self-foaming cleansing composition, and it could be preferred to be present in an amount of from 0.2 to 0.4% by weight, based on the total weight of the self-foaming cleansing composition, for example, 0.2 to 0.3%, including any and all ranges and values subsumed therein.

The butter can be aloe butter, avocado butter, bacuri butter, cocoa butter, coconut butter, coffee bean butter, cupuagu butter, hemp seed butter, illipe butter, kokum butter, macadamia nut butter, mango butter, mochacchino butter, murumuru butter, olive butter, pistachio nut butter, refined butter, shea butter, sweet almond butter, tucuma butter, ucuuba butter, or a combination thereof.

The wax can be carnauba, spermaceti, beeswax, lanolin, and derivatives thereof.

Other useful skin benefit agents include the following:
(a) silicone oils and modifications thereof such as linear and cyclic polydimethylsiloxanes; amino, alkyl, alkylaryl, and aryl silicone oils;
(b) fats and oils including natural fats and oils such as soybean, rice bran, persic, and mink oils; cacao fat; beef tallow and lard; hardened oils obtained by hydrogenating the aforementioned oils; and synthetic mono, di and triglycerides such as myristic acid glyceride and 2-ethylhexanoic acid glyceride;
(c) hydrophobic and hydrophilic plant extracts;
(d) hydrocarbons such as liquid paraffin, petrolatum, microcrystalline wax, ceresin, squalene, pristan and mineral oil;
(e) higher fatty acids such as lauric, myristic, palmitic, stearic, behenic, oleic, linoleic, linolenic, lanolic, isostearic, arachidonic and poly unsaturated fatty acids (PUFA);
(f) higher alcohols such as lauryl, cetyl, stearyl, oleyl, behenyl, cholesterol and 2-hexydecanol alcohol;
(g) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol monolaurate, glycerol distearate, glycerol tristearate, alkyl lactate, alkyl citrate and alkyl tartrate;
(h) essential oils and extracts thereof such as mentha, jasmine, camphor, white cedar, bitter orange peel, ryu, turpentine, cinnamon, bergamot, citrus unshiu, calamus, pine, sugar cane, chamomile, yarrow, liquorice, lavender, bay, clove, hiba, eucalyptus, lemon, starflower, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils;
(i) polyhydric alcohols, for example, glycerine, sorbitol, propylene glycol, and the like; and polyols such as the polyethylene glycols, examples of which are: Polyox WSR-205 PEG 14M, Polyox WSR-N-60K PEG 45M, or Polyox WSR-N-750, and PEG 7M;
(j) lipids such as cholesterol, ceramides, sucrose esters and pseudo-ceramides as described in European Patent Specification No. 556,957;
(k) vitamins, minerals, and skin nutrients such as milk, vitamins A, E, and K; vitamin alkyl esters, including vitamin C alkyl esters; magnesium, calcium, copper, zinc and other metallic components;
(l) sunscreens such as octyl methoxyl cinnamate (Parsol MCX) and butyl methoxy benzoylmethane (Parsol 1789);
(m) phospholipids; and
(n) anti-aging compounds such as alpha-hydroxy acids and beta-hydroxy acids.

Skin benefit agents commonly account for up to 30% by weight of the self-foaming cleansing composition, with levels of 0 to 25% by weight preferred, more particularly 0 to 20% by weight, being typical of the levels at which those skin benefit agents are employed in many of the subject formulations. Preferred skin benefit agents include fatty acids, hydrocarbons, polyhydric alcohols, polyols and mixtures thereof, with emollients that include at least one C₁₂ to C₁₈ fatty acid, petrolatum, glycerol, sorbitol and/or propylene glycol being of particular interest in one or more embodiments.

Other optional ingredients include water soluble/dispersible polymers. These polymers can be cationic, anionic, amphoteric or nonionic types with molecular weights higher than 100,000 Dalton. They are known to increase the viscosity and stability of liquid personal cleansing formulation, to enhance in-use and after-use skin sensory properties, and to enhance lather creaminess and lather stability. When present, the total amount of such polymers commonly present in the self-foaming cleansing compositions is 0.1 to 10% by weight, based on the total weight of the self-foaming cleansing composition.

Examples of water soluble or dispersible polymers include the carbohydrate gums such as cellulose gum, microcrystalline cellulose, cellulose gel, hydroxyethyl cellulose, hydroxypropyl cellulose, sodium carboxymethylcellulose, methyl cellulose, ethyl cellulose, guar gum, gum karaya, gum tragacanth, gum arabic, gum acacia, gum agar, xanthan gum and mixtures thereof; modified and nonmodified starch granules and pregelatinized cold water soluble starch; emulsion polymers such as ACUYLN® 28, ACULYLN® 22 or CARBAPOL® Aqua SF1; cationic polymer such as modified polysaccharides including cationic guar available from Rhone Poulenc under the trade name JAGUAR™ C13S, JAGUAR™ C14S, JAGUAR™ 017, or JAGUAR™ 016 cationic modified cellulose such as UCARE™ Polymer JR 30 or JR 40 from Amerchol; N-HANCE®3000, N-HANCE® 3196, N-HANCE® GPX 215 or N-HANCE® GPX 196 from Hercules; synthetic cationic polymer such as MERQUAT® 100, MERQUAT® 280, MERQUAT® 281 and MERQUAT® 550 sold by Nalco; cationic starches such as STALOK® 100, 200, 300 and 400 sold by Staley Inc.; cationic galactomannans such as GALACTASOL® 800 series by Henkel, Inc.; QUADROSOFT® LM-200; and Polyquaternium-24. Also suitable are high molecular weight polyethylene glycols such as POLYOX® WSR-205 (PEG 14M), POLUOX® WSR-N-60K (PEG 45), and POLYOX® WSR-301 (PEG 90M).

An opacifier may be optionally present in the self-foaming cleansing composition. When opacifiers are present, the composition is generally opaque. Examples of opacifiers include titanium dioxide, zinc oxide, and the like. A particularly preferred opacifier that can be employed when an opaque soap composition is desired is ethylene glycol mono- or di-stearate, for example in the form of a 20% solution in sodium lauryl ether sulphate. An alternative opacifying agent is zinc stearate.

The product can take the form of a water-clear, i.e., transparent composition, in which case it will not contain an opacifier.

Desirably the optional skin benefit agents used in the antimicrobial composition disclosed herein include niacinamide (vitamin B₃), tocopherol (Vitamin E), aloe vera, alpha-hydroxy acids and esters, beta-hydroxy acids and esters, hydroxyethyl urea, polyhydroxy acids and esters, creatine, hydroquinone, t-butyl hydroquinone, mulberry, hyaluronic acid and salts thereof (including, but not limited to, Na+ and K+ salts of the same), extract, liquorice extract, resorcinol derivatives, or a combination thereof. For example, the skin benefit agent can be sodium hyaluronate. Such benefit agents, including sodium hyaluronate can be present in an amount of 0.0001 to 10%, for example, 0.001 to 6.5%, for example, 0.01 to 3.5%, and for example, 0.01% by weight, based on total weight of the self-foaming cleansing composition including any and all values and ranges subsumed therein.

Further optional water-soluble skin benefit agents include acids, such as amino acids like arginine, valine or histidine. Other vitamins can be used such as vitamin B₂, picolinamide, panthenol (vitamin B₅), vitamin Br, vitamin C, a combination thereof or the like. Derivatives (generally meaning something that has developed or been obtained from something else), and especially, water soluble derivatives of such vitamins can also be employed. For instance, vitamin C derivatives such as ascorbyl tetraisopalmitate, magnesium ascorbyl phosphate and ascorbyl glycoside may be used alone or in combination with each other. Niacinamide derivatives such as nicotinamide adenine dinucleotide (NADH) and nicotinamide adenine dinucleotide phosphate (NADPH) may be used alone or in combination with each other. Electrolytes such as NaCl and/or KCl, MgCl₂ may also be used. The total amount of optional water-soluble benefit agents (including mixtures) when present in the composition disclosed herein can be 0.0001 to 10%, preferably, 0.001 to 6.5%, and most preferably, 0.01 to 3.5% by weight, based on total weight of the self-foaming cleansing composition, including any and all values and ranges subsumed therein.

It is also within the scope of the self-foaming cleansing composition to optionally include oil soluble benefit agents. Illustrative examples of the types of oil soluble benefit agents that can optionally be used in the self-foaming cleansing composition disclosed herein include components such as vitamins like vitamin A, D, E (tocopherol) and K (and their oil soluble derivatives).

A particularly preferred oil soluble benefit agent includes Vitamin E. When used, Vitamin E can be present in an amount of 0.001 to 0.1% by weight, based on the total weight of the self-foaming cleansing composition, for example, 0.005 to 0.09% by weight, based on the total weight of the self-foaming cleansing composition, for example, 0.009 to 0.05% by weight, based on the total weight of the self-foaming cleansing composition, for example, 0.01 to 0.04% by weight, based on the total weight of the self-foaming cleansing composition, for example, 0.01 to 0.04% by weight, based on the total weight of the self-foaming cleansing composition, including any and all ranges and values subsumed therein.

Other optional oil soluble benefit agents for use include resorcinols and resorcinol derivatives like 4-hexyl resorcinol, 4-phenylethyl resorcinol, 4-cyclopentyl resorcinol, 4-cyclohexyl resorcinol 4-isopropyl resorcinol or a combination thereof. Also, 5-substituted resorcinols like 4-cyclohexyl-5-methylbenzene-1,3-diol, 4-isopropyl-5-methylbenzene-1,3-diol, combination thereof or the like may be used. The 5-substituted resorcinols and their synthesis are described in commonly assigned U.S. Pat. No. 10,470,986 B2.

Even other oil soluble benefit agents that can be used include omega-3 fatty acids, omega-6 fatty acids, climbazole, magnolol, honokiol, farnesol, ursolic acid, myristic acid, geranyl geraniol, oleyl betaine, cocoyl hydroxyethyl imidazoline, hexanoyl sphingosine, 12-hydroxystearic acid (12HSA), petroselinic acid, conjugated linoleic acid, stearic acid, palmitic acid, lauric acid, terpineol, thymol essential components, the dissolution auxiliary selected from limonene, pinene, camphene, cymene, citronellol, citronellal, geraniol, nerol, linalool, rhodinol, borneol, isoborneol, menthone, camphor, safrole, isosafrole, eugenol, isoeugenol, tea tree oil, eucalyptus oil, peppermint oil, neem oil, lemon grass oil, orange oil, bergamot oil, or a combination thereof.

Another optional oil soluble benefit agent that may be used is a retinoic acid precursor. The retinoic acid precursor can be retinol, retinal, retinyl ester, retinyl propionate, retinyl palmitate, retinyl acetate or a combination thereof. Retinyl propionate, retinyl palmitate and combinations thereof are typically preferred. Still another retinoic acid precursor for use is hydroxyanasatil retinoate made commercially available under the name RETEXTRA® as supplied by Molecular Design International. The same may be used in a combination with any of the oil soluble benefit agents described herein.

When an optional (i.e., 0.0 to 1.5% by weight based on the total weight of the self-foaming cleansing composition) oil soluble benefit agent is used, it typically is present in an amount of 0.001 to 1.5% by weight of the overall self-foaming cleansing composition including any and all values and ranges subsumed therein, and for example, 0.05 to 1.2% by weight, for example, 0.2 to 0.5% by weight of the total weight of the self-foaming cleansing composition.

Preferred skin benefit agents include fatty acids, hydrocarbons, polyhydric alcohols, polyols, and mixtures thereof, with emollients that include at least one C₁₂ to C₁₈ fatty acid, petrolatum, glycerol, sorbitol, and/or propylene glycol being of particular interest in one or more embodiments. The agents may be added at an appropriate step during the process of making the self-foaming cleansing composition. Some benefit agents may be introduced as macro domains.

Other optional ingredients like antioxidants, perfumes, polymers, colorants, deodorants, dyes, enzymes, foam boosters, germicides, anti-microbials, lathering agents, pearlescers, skin conditioners, stabilizers, or superfatting agents, may be added in suitable amounts in the process of making the self-foaming cleansing composition. Sodium metabisulphite, ethylene diamine tetra acetic acid (EDTA), borax, or ethylene hydroxy diphosphonic acid (EHDP) can be added to the formulation.

The self-foaming cleansing composition disclosed herein can optionally be used to deliver antimicrobial benefits. Antimicrobial agents that can be included to deliver these benefits include oligodynamic metals or compounds thereof. Preferred metals are silver, copper, zinc, gold, aluminum, or a. In the ionic form it may exist as a salt or any compound in any applicable oxidation state. Preferred silver compounds are silver oxide, silver nitrate, silver acetate, silver sulfate, silver benzoate, silver salicylate, silver carbonate, silver citrate, silver phosphate, or a combination thereof, with silver oxide, silver sulfate and silver citrate being of particular interest in one or more embodiments. In at least one aspect, the silver compound is silver oxide. Oligodynamic metal or a compound thereof can be included in an amount of 0.0001 to 2%, preferably 0.001 to 1% by weight of the cleansing bar composition. Alternately an essential oil antimicrobial active may be included in the cleansing bar composition. Essential oil actives which can be included are terpineol, thymol, carvacol, (E)-2(prop-1-enyl) phenol, 2-propylphenol, 4-pentylphenol, 4-sec-butylphenol, 2-benzyl phenol, eugenol, or a combination thereof. Furthermore, preferred essential oil actives are terpineol, thymol, carvacrol, thymol, or a combination thereof, with the most preferred being terpineol or thymol, or a combination thereof. When present, essential oil actives can be included in an amount of 0.001 to 1%, preferably 0.01 to 0.5% by weight of the composition.

Even other ingredients which may be used include octopirox (piroctone), zinc pyrithione, chloroxylenol, triclosan, cetylpyridinium chloride, as well as silver compounds including silver oxide, nitrate, sulfate, phosphate, carbonate, acetate, benzoate, a combination thereof or the like. If used, these other components typically make up from 0.001 to 1.6% by weight of the overall self-foaming cleansing composition including any and all values and ranges subsumed therein, and preferably, from 0.01 to 1.2% by weight.

Except where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration or amount, any particular upper concentration can be associated with any particular lower concentration or amount as well as any subranges consumed therein. In that regard, it is noted that all ranges disclosed herein are inclusive of the endpoints, and the endpoints are independently combinable with each other (e.g., ranges of “up to 25% by weight, or, more specifically, 5% by weight to 20% by weight, in inclusive of the endpoints and all intermediate values of the ranges of 5% by weight to 25% by weight, etc.). “Combination is inclusive of blends, mixtures, alloys, reaction products, and the like. Furthermore, the terms “first”, “second”, and the like herein do not denote any order, quantity, or importance, but rather are used to distinguish one element from another. The terms “a” and “an” and “the” herein do not denote a limitation of quantity and are to be construed to cover both the singular and the plural, unless otherwise indicated herein or clearly contradicted by context. The suffix “(s)” as used herein is intended to include both the singular and the plural of the term it modifies, thereby including one or more of the term (e.g., the film(s) includes one or more films). Reference throughout the specification to “one embodiment”, “one aspect”, “another embodiment”, “another aspect”, “an embodiment”, “an aspect” and so forth means that a particular element (e.g., feature, structure, and/or characteristic) described in connection with the embodiment or aspect is included in at least one embodiment or aspect described herein and may or may not be present in other embodiments or aspects. In addition, it is to be understood that the described elements may be combined in any suitable manner in the various embodiments or aspects.

All cited patents, patent applications, and other references are incorporated herein by reference in their entirety. However, if a term in the present application contradicts or conflicts with a term in the incorporated reference, the term from the present application takes precedence over the conflicting term from the incorporated reference. While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

For the avoidance of doubt the word “comprising” is intended to mean “including” but not necessarily “consisting of” or “composed of.” In other words, the listed steps, options, or alternatives need not be exhaustive.

The disclosure of the invention as found herein is to be considered to cover all aspects as found in the claims as being multiply dependent upon each other irrespective of the fact that claims may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated. The various features of the present invention referred to in individual sections above apply, as appropriate, to other sections mutatis mutandis.

Consequently, features specified in one section may be combined with features specified in other sections as appropriate. Any section headings are added for convenience only and are not intended to limit the disclosure in any way.

### EXAMPLES

The following examples are merely illustrative of the self-foaming cleansing composition disclosed herein and are not intended to limit the scope hereof.

### Example 1: Gene Expression

In the following samples, compositions were made with varying levels of moringa seed oil and tested in a living skin equivalent assay. The base composition was tested with levels of 0.1% by weight, 0.2% by weight, and 0.3% by weight of moringa seed oil, based on the total weight of the self-foaming cleansing composition. The compositions are shown in Table 1. The living skin equivalent assay study monitored gene expression of several markers after application of the test solutions to the living skin equivalent assay.

TABLE 1
Moringa Seed Oil Living Skin Equivalent Assay Compositions

| | Material | | | |
|---|---|---|---|---|
| | % by weight in composition | | | |
| | Sample # | | | |
| | | Sample 1 | Sample 2 | Sample 3 |
| | Comparative | 0.1% | 0.2% | 0.3% |
| | Example 3 | moringa | moringa | moringa |
| | Base | seed oil | seed oil | seed oil |
| DI Water | q.s. | q.s. | q.s. | q.s. |
| Citric Acid | 0.500% | 0.500% | 0.500% | 0.500% |
| Polysorbate | 0.750% | 0.750% | 0.750% | 0.750% |
| 20 | | | | |
| Moringa | 0.000% | 0.100% | 0.200% | 0.300% |
| Seed Oil | | | | |
| Glycerol | 10.50% | 10.50% | 10.50% | 10.50% |
| CAPB | 3.00% | 3.00% | 3.00% | 3.00% |
| Sodium | 2.50% | 2.50% | 2.50% | 2.50% |
| Lauroyl | | | | |
| Glutamate | | | | |

Comparative Example 1 was a non-treated control meaning no application on the skin model. Comparative Example 2 was another control where phosphate-buffered saline (PBS) was applied topically to the skin model.

TABLE 2
Living Skin Equivalent Results

| | IL-1a | CASP14 | AQP3 |
|---|---|---|---|
| Sample # | (pg/ml*) | (Fold change) | (Fold change) |
| Comparative | 30.03 ± 5.49 | 1.02 ± 0.19 | 1.00 ± 0.10 |
| Example 1 | | | |
| Comparative | 367.64 ± 2.54 | 0.53 ± 0.47 | 0.28 ± 0.19 |
| Example 2 | | | |
| SQOOH | 483.84 ± 11.42 | 0.54 ± 0.31 | 0.27 ± 0.04 |
| Comparative | 301.35 ± 28.54 | 1.87 ± 0.69 | 1.26 ± 0.15 |
| Example 3 | | | |
| Sample 1 | 307.78 ± 25.01 | 0.46 ± 0.27 | 0.48 ± 0.29 |
| Sample 2 | 191.02 ± 47.80 | 0.80 ± 0.61 | 0.27 ± 0.02 |
| Sample 3 | 195.60 ± 7.14 | 4.51 ± 0.90 | 1.18 ± 0.32 |

*pg/ml = picogram/milliliter

As can be seen in Table 2, the composition with 0.3% by weight moringa seed oil showed significant impacts on the CASP14, ASP3, and IL1-α genes that are responsible natural moisturizing factor processing, skin hydration, and inflammation respectively, when compared to a squalene monoperoxide (SQOOH) control. The composition with 0.2% by weight moringa seed oil increased gene expression in proteins related to skin hydration (HAS3) and barrier lipids (CERS and ELOVL2). These results demonstrated that moringa seed oil is multifunctional in a rinse-off cleansing composition.

### Example 2: Antioxidation

To add antioxidation properties to the self-foaming cleansing composition, tocopherol (Vitamin E) was added to the composition of Sample 3 at 0.035% by weight (Sample 4) and 0.0175% by weight (Sample 5). Antioxidation properties were then tested.

Table 3 lists the formulations for Samples 4 and 5.

TABLE 3
Samples 4 and 5 formulations

Material
% by weight in composition
Sample #

| | Sample 4 | Sample 5 |
|---|---|---|
| | 0.3% moringa | 0.3% moringa |
| | seed oil | seed oil |
| DI Water | q.s. | q.s. |
| Citric Acid | 0.500% | 0.500% |
| Polysorbate 20 | 0.750% | 0.750% |
| Moringa Seed | 0.200% | 0.300% |
| Oil | | |
| Glycerol | 10.50% | 10.50% |
| CAPB | 3.00% | 3.00% |
| Sodium Lauroyl | 2.50% | 2.50% |
| Glutamate | | |
| Tocopherol | 0.035% | 0.0175% |
| (Vitamin E) | | |

TABLE 4
Antioxidation Results

| | Dilutions | % Reduction over | |
|---|---|---|---|
| | (diluted in ethanol) | DPPH reagent control | SD* |
| Vitamin C 0.25% | | 95.04 | 1.71 |
| (Positive control) | | | |
| Sample 4 | 50% (1:1) | 86.98 | 3.02 |
| | 10% (1:10) | 82.16 | 3.43 |
| | 1% (1:100) | 31.42 | 3.81 |
| | 0.1% (1:1000) | 1.94 | 5.46 |
| Sample 5 | 50% (1:1) | 79.03 | 0.66 |
| | 10% (1:10) | 69.60 | 2.32 |
| | 1% (1:100) | 7.37 | 0.95 |
| | 0.1% (1:1000) | 1.72 | 1.96 |

*SD = standard deviation

The antioxidant activity of the formulations in Samples 4 and 5 were tested using the DPPH scavenging method (1,1-Diphenyl-2-picrylhydrazyl). DPPH is a stable free radical. On accepting hydrogen from a corresponding donor, its solutions lose the characteristic deep purple color (λmax 515-517 nanometers (nm)). The antiradical activity of Samples 4 and 5 was expressed as a percentage decrease of DPPH concentration. When the formulas for Samples 4 and 5 were tested at varying ethanol dilutions of 0.1% (0.1% formula, 99.9% ethanol) to 50% (50% formula, 50% ethanol), there was a dose dependent response to antioxidant activity. When dosed with tocopherol (Vitamin E) at 0.035% (Sample 4) and 0.0175% (Sample 5), there was a statistically significant carry of free radical reduction at dilutions down to 10%.

1. A self-foaming cleansing composition, comprising:
2.5 to 10% by weight of a surfactant based on the total weight of the self-foaming cleansing composition, wherein the surfactant comprises an anionic surfactant and a zwitterionic surfactant;
5 to 75% by weight of a humectant, based on the total weight of the cleansing composition; and
0.01 to 1.0% by weight of an oil emollient based on the total weight of the self-foaming cleansing composition, wherein the oil emollient comprises an oil comprising Baobab seed oil, Argan kernel oil, behenyl neopentanoate, Borage seed oil, Rapeseed seed oil, Tamanu seed oil, False Flax seed oil, Camellia seed oil, caprylic/capric triglyceride, Safflower seed oil, coco-caprylate/caprate, Coconut oil, Hazel seed oil, Crambe seed oil, Cotton seed oil, Sunflower seed oil, Sea Buckthorn oil, isopropyl myristate, isononyl isonanonoate, isopropyl palmitate, jojoba esters, lanolin oil and lanolin derivatives, Limnanthes Alba (Meadowfoam) seed oil, Linseed seed oil, Macadamia seed oil, Moringa seed oil, Evening Primrose oil, Olive fruit oil, Babassu seed oil, Rice germ oil, Avocado oil, Sacha inchi seed oil, Sweet Almond oil, Castor seed oil, Rosehip seed oil, Chia seed oil, Sage oil, Mongongo kernel oil, Marula seed oil, Sesame seed oil, Sal seed oil, silicone, Jojoba seed oil, squalane, Thyme oil, algae oil, Wheat germ oil, Grapeseed oil, Corn oil, or a combination thereof;
wherein the composition is essentially free of sulfate;
wherein the composition is biodegradable.

2. The self-foaming cleansing composition of claim 1, wherein the surfactant comprises the anionic surfactant and the amphoteric surfactant in a weight ratio of 0.5:2.

3. The self-foaming cleansing composition of claim 1, wherein the anionic surfactant comprises sodium lauroyl glycinate, sodium cocoyl glycinate, sodium lauroyl glutamate, sodium cocoyl glutamate, sodium lauroyl isethionate, sodium cocoyl isethionate, alpha olefin sulfonate (AOS), or a combination thereof.

4. The self-foaming cleansing composition of claim 1, wherein the zwitterionic surfactant comprises cocodimethyl carboxymethyl betaine, cocamidopropyl betaine, laurylamidopropyl betaine, cocamidopropyl hydroxysultaine, lauryl hydroxysultaine, cocamide monoethanolamide, or a combination thereof.

5. The self-foaming cleansing composition of claim 1, wherein the humectant comprises glycerol, propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol, or a combination thereof.

6. (canceled)

7. (canceled)

8. (canceled)

9. (canceled)

10. The self-foaming cleansing composition of claim 1, further comprising 0.01 to 1.0% by weight of a chelator based on the total weight of the self-foaming cleansing composition wherein the chelator comprises sodium gluconate, nitrilotriacetic acid (NTA), ethylene diamine disuccinic acid (EDDS), iminodisuccinic acid (IDS), methylglycinediacetic acid (MGDA), L-glutamic acid N,N-diacetic acid (GLDA), ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), sodium citrate, salts of methyglycinediacetic acid, or a combination thereof.

11. The self-foaming cleansing composition of claim 1, further comprising a nourishing agent, wherein the nourishing agent comprises 10-hydroxystearic acid, 12-hydroxystearic acid, petroselinic acid, conjugated linoleic acid, lauric acid, myristic acid, palmitic acid, stearic acid, behenic acid, or a combination thereof.

12. (canceled)

13. (canceled)

14. (canceled)

15. (canceled)

16. The self-foaming cleansing composition of claim 2, wherein the weight ratio is 0.75 to 1.5.

17. The self-foaming cleansing composition of claim 2, wherein the weight ratio is 0.8 to 1.

18. The self-foaming cleansing composition of claim 2, wherein the weight ratio is 0.85 to 0.95.
